(19) United States
(12) Patent Application Publication
(10) Pub. No.: US 2025/0186473 A1
(43) Pub. Date: Jun. 12, 2025

(54) Compositions and Methods for Treating Kidney Disease

(71) Applicant: Neukidney Incorporated, Arcola, TX (US)

(72) Inventor: Ebima Clifford Okundaye, Arcola, TX (US)

(21) Appl. No.: 18/969,905

(22) Filed: Dec. 5, 2024

Related U.S. Application Data
(60) Provisional application No. 63/608,130, filed on Dec. 8, 2023.

(51) Int. Cl. A61K 31/706; A61K 31/122; A61K 31/197; A61K 31/522; A61P 13/12

(52) U.S. Cl. CPC A61K 31/706; A61K 31/122; A61K 31/197; A61K 31/522; A61P 13/12

(57) ABSTRACT

The present disclosure relates generally to compositions and methods for treating chronic kidney disease.

### CROSS-REFERENCE TO RELATED APPLICATIONS

This application claims priority to U.S. Provisional Application No. 63/608,130, filed Dec. 8, 2023, which is incorporated by reference herein in its entirety.

### BACKGROUND OF THE DISCLOSURE

### Field of the Invention

This disclosure relates generally to compositions and methods of treating of kidney disease.

### Description of Related Art

Chronic kidney disease (CKD) is estimated to affect nearly over 800 million people globally today (with roughly 125,000 people ending up on dialysis annually in the United States alone (Webster A., et al. Lancet 2017; Hill N R, et al. PLOS One 2016). CKD is a contributor to illness and is associated with a diminished quality of life and reduced life expectancy (Hill N R, et al. PLOS One 2016; Hiddo J. L. et al. N Engl J Med 2020; Weiner D E, et al. J Am Soc Nephrol. 2004; Tonelli M, et al. J Am Soc Nephrol. 2006). CKD's impact on comorbidities and incidence mortality poses a challenging difficulty in addressing this global disease.

While other causes are minor incidence etiology, diabetes and hypertension are two of the most common causes of CKD (Chen T K, et al. JAMA 2019). Several studies have proposed and evaluated ways of curbing the progression of the disease. Lowering blood pressure (Mahmoodi B K, et al. Lancet 2012; Go A S, et al. N Engl J Med. 2004; Ettehad D, et al. Lancet 2016) and the widespread use of Angiotensin-converting enzyme inhibitors (ACEI) or Angiotensin II Receptor Blockers (ARB) to block the endothelial damage via the Renin-Angiotensin-Aldosterone System (RAAS) and cause a decrease in intraglomerular pressure have been shown to be a means of slowing the progression of the disease especially with strong evidence in patients with diabetes (Qin Y, et al. Pharmacoepidemiol Drug Saf. 2016). The use of bicarbonate supplementation has also been shown to slow the rate of kidney decline (de Brito-Ashurst I, et al. J. Am Soc Nephrol. 2009; Mahajan A, et al. Kidney Int. 2010; Phisitkul S, et al. Kidney Int. 2010). These therapies, along with a more recently discovered sodium glucose cotransporter inhibition (SGLT2) via its natriuresis, and glucose induce osmotic diuresis and subsequent intraglomerular pressure reduction have been helpful in slowing the rate of decline in patients with or without diabetic nephropathy (Wanner C, et al. N Engl J Med 2016; Neal B, et al. N Engl J Med 2017; Wiviott S D, et al. N Engl J Med 2019).

Some studies have proposed mechanisms of ongoing pathophysiological pathways of the disease with the presence of proteinuria directly from or along with intraglomerular pressure increase are correlated with a more rapid rate of kidney damage and progressive decline in glomerular function (Cravedi P, et al. Br J Clin Pharmacol. 2013). Another potential mechanism of renal decline has been proposed to occur by intracellular mechanisms including decreased intracellular Nicotinamide adenine dinucleotide (NAD+) levels and dysregulation of oxidative processes with secondary inflammatory marker release, which suggests mitochondrial oxidative phosphorylation changes within the tubular and interstitial cells present in the kidney (Morevati M, et al. Int J Mol Sci. 2022). NAD+ supplies energy for deoxidation and anti-inflammatory reactions fostering the production of adenosine triphosphate (ATP). NAD molecules and their activity in electron transfer in renal tissue has been shown to be low in CKD states. This cascade of oxidative inflammatory cell markers has been shown to contribute to renal fibrosis (Genovese F., et al. Fibrogenesis Tissue Repair. 2014; Sparding N., et al. Kidney 360. 2022; Humphreys B. D. Annu. Rev. Physiol. 2018). Mitochondrial dysfunction has also been observed in CKD, and preventing mitochondrial damage reduces fibrogenesis in rat models (Jiang M., et al. Am. J. Physiol. Renal. Physiol. 2020).

An earlier animal study has shown that oral supplementation with Nicotinamide (NAM) a precursor of NAD+ attenuated progression of CKD in rat models (Kumakura S, et al. Toxins (Basel). 2021). There was a significant difference in BUN and serum creatinine changes as well as histological changes of decreased fibrosis in kidney tissues of NAM supplemented rats. Decreased availability or impaired NAD production has been linked to premature renal aging in some studies. These studies strongly suggested NAD+ administration or activity optimization may be a novel therapeutic approach for CKD prevention (Kumakura S, et al. Toxins (Basel). 2021; Chanvillard L, et al. Cells. 2022; Liu X, et al. Front Physiol. 2021; Doke T, et al. Nat Metab. 2023). However, the target of this NAD+ supplementation did not show any statistical benefit in some CKD patients in a combined trial (Chanvillard L, et al. Cells. 2022).

Although some of the aforementioned medications have been shown to be helpful in minimizing the rate of CKD progression, there is currently no therapeutic option available for patients to restore or repair any already incident loss of glomerular function. Thus, there is a need for therapeutics for attenuating the progression of CKD.

### BRIEF SUMMARY OF THE DISCLOSURE

The following embodiments and aspects thereof are described and illustrated in conjunction with compositions and methods which are meant to be exemplary and illustrative, not limiting in scope.

Disclosed herein are compositions and methods for treating chronic kidney disease.

In a first aspect, the present disclosure provides a pharmaceutical composition of a therapeutically effective amount of an anti-inflammatory agent, an ubiquinol and/or citric acid/sodium citrate, and a nicotinamide oxygenase modifier. Also contemplated is a NAD precursor in lieu of the nicotinamide oxygenase modifier.

In an embodiment of the first aspect, the anti-inflammatory modifier is pentoxifylline. In another embodiment of the first aspect, the ubiquinone, or its reduced form ubiquinol, is coenzyme Q10. In a further embodiment of the first aspect, the NAD oxygenase modifier is acetylcysteine. In yet another embodiment, the NAD precursor is nicotinamide mononucleotide.

In an embodiment of the first aspect, the therapeutically effective amount of pentoxifylline is about 400 mg to about 800 mg. In another embodiment of the first aspect, the therapeutically effective amount of CoQ10 is about 100 mg to about 200 mg. In another embodiment of the first aspect, the therapeutically effective amount of citric acid/sodium citrate is about 3000 mg-2004 mg or about 6000 mg-4008 mg. In a further embodiment of the first aspect, the therapeutically effective amount of acetylcysteine is about 1200 mg to about 2400 mg. In yet another embodiment, of the first aspect, the therapeutically effective amount of nicotinamide mononucleotide is about 1000 mg to about 2000 mg.

In an embodiment of the first aspect, the composition further comprises one or more of a pharmaceutically suitable carrier, dilutant, and/or excipient. In another embodiment the composition further comprises a sweetener or a flavorant.

In an embodiment of the first aspect, the composition is a powder or mixture of pills. In another embodiment of the first aspect, the composition is formulated for oral administration, injection, intravenous (IV), or intramuscular delivery.

In a second aspect, the present disclosure provides a method of treating chronic kidney disease in a subject by administering a therapeutically effective dose of the disclosed pharmaceutical composition. The contemplated composition comprises (i) a pentoxifylline; (ii) coenzyme Q10 and/or citric acid/sodium citrate; (iii) acetylcysteine or nicotinamide mononucleotide. The disclosed method of treating a subject with the composition increases estimated glomerular filtration rate (eGFR) and/or reduces proteinuria levels in the subject.

In an embodiment of the second aspect, the method improves eGFR by at least 5 mL per minute. In another embodiment of the second aspect, the subject has proteinuria levels in the either non-nephrotic or nephrotic proteinuria range.

In an embodiment of the second aspect, the pharmaceutical composition is delivered to the subject by oral administration, injection, intravenously, or intramuscularly. In a specific embodiment of the second aspect, the pharmaceutical composition is administered orally.

In an embodiment of the second aspect, the pharmaceutical composition is given at least once a day. In another embodiment of the second aspect, the pharmaceutical composition is a powder or mixture of pills. In yet another embodiment of the second aspect, the pharmaceutical composition is administered daily for at least 3 months.

In one embodiment of the second aspect, renal function improves by about 20% in subjects with stage 3, stage 4, or stage 5 chronic kidney disease.

In a third aspect, the present disclosure provides a method of treating chronic kidney disease in a subject. The method comprises administering a therapeutically effective dose of a pharmaceutical composition to the subject. The contemplated composition comprises (i) about 400 mg to about 800 mg of pentoxifylline; (ii) about 100 mg to about 200 mg of coenzyme Q10 and/or about 3000 mg-2004 mg to about 6000 mg-4008 mg citric acid/sodium citrate; and (iii) about 1200 mg to about 2400 mg of acetylcysteine or 1000 mg to about 2000 mg of nicotinamide mononucleotide. The contemplated composition further comprises (i) one or more of a pharmaceutically suitable carrier, dilutant, and/or excipient; and (ii) and sweetener or a flavorant (e.g., a natural and/or artificial sweetener or flavorant). The contemplated method comprises at least one of increasing estimated glomerular filtration rate (eGFR) and/or reducing proteinuria levels in the subject.

In an embodiment of the third aspect, the pharmaceutical composition is administered daily for at least 3 months. In another embodiment of the third aspect, administration of the pharmaceutical composition improves eGFR by at least 5 mL per minute. In yet another embodiment of the third aspect, renal function improves by about 20% in subjects with stage 3, stage 4, or stage 5 chronic kidney disease.

In addition to the exemplary aspects and embodiments described above, further aspects and embodiments will become apparent by reference to the drawings and by study of the following detailed description.

### BRIEF DESCRIPTION OF THE DRAWINGS

The accompanying drawings are included to provide a further understanding of the methods and compositions of the disclosure and are incorporated in and constitute a part of this specification. The drawings illustrate one or more non-limiting embodiment(s) of the disclosure, and together with the description serve to explain the principles and operation of the disclosure.

FIGS. 1A-1B show graphs of mean renal function with drug treatment (also referred to herein as QRX or QRX3). FIG. 1A shows a line graph of mean renal function in patients treated with the disclosed pharmaceutical composition over different time periods via estimated glomerular rate (eGFR) by Modification of Diet in Renal Disease (MDRD). FIG. 1B shows a bar graph of mean renal function in patients treated with the disclosed pharmaceutical composition over different time periods via estimated glomerular rate (eGFR) by Modification of Diet in Renal Disease (MDRD).

FIG. 2 shows a bar graph of the pattern of mean improvement of eGFR for all CKD stages based on the level of baseline proteinuria (Nephrotic vs non nephrotic) and their percentage mean change.

FIG. 3 shows a bar graph of the pattern of improvement of eGFR by percentage at 3 and 6 months after treatment when compared to baseline for patients in CKD stage 3.

FIG. 4 shows a bar graph of the pattern of improvement of eGFR by percentage at 3 and 6 months after treatment when compared to baseline for patients in CKD stage 4.

FIG. 5 shows a bar graph of the pattern of improvement of eGFR by percentage at 3 and 6 months after treatment when compared to baseline for patients in CKD stage 5.

### DETAILED DESCRIPTION OF THE DISCLOSURE

All publications, including but not limited to patents and patent applications, cited in this specification are herein incorporated by reference as though set forth in their entirety in the present application.

As utilized in accordance with the present disclosure, unless otherwise indicated, all technical and scientific terms shall be understood to have the meaning commonly understood by one of ordinary skill in the art. Unless otherwise required by context, singular terms shall include the plural and plural terms shall include the singular.

Throughout this specification, unless the context specifically indicates otherwise, the terms “comprise” and “include” and variations thereof (e.g., “comprises,” “comprising,” “includes,” and “including”) will be understood to indicate the inclusion of a stated component, feature, element, or step or group of components, features, elements or steps but not the exclusion of any other component, feature, element, or step or group of components, features, elements, or steps. Any of the terms “comprising,” “consisting essentially of,” and “consisting of” may be replaced with either of the other two terms, while retaining their ordinary meanings.

As used herein, the singular forms “a,” “an,” and “the” include plural referents unless the context clearly indicates otherwise.

In some embodiments, percentages disclosed herein can vary in amount by +10, 20, or 30% from values disclosed and remain within the scope of the contemplated disclosure.

Unless otherwise indicated or otherwise evident from the context and understanding of one of ordinary skill in the art, values herein that are expressed as ranges can assume any specific value or sub-range within the stated ranges in different embodiments of the disclosure, to the tenth of the unit of the lower limit of the range, unless the context clearly dictates otherwise.

As used herein, ranges and amounts can be expressed as “about” a particular value or range. About also includes the exact amount. For example, “about 5%” means “about 5%” and also “5%.” The term “about” can also refer to ±10% of a given value or range of values. Therefore, about 5% also means 4.5%-5.5%, for example.

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.”

“Pharmaceutically acceptable” refers to those compounds, materials, compositions, and/or dosage forms which are, within the scope of sound medical judgment, suitable for contact with the tissues of human beings and animals without excessive toxicity, irritation, allergic response, or other problems or complications commensurate with a reasonable benefit/risk ratio or which have otherwise been approved by the United States Food and Drug Administration as being acceptable for use in humans or domestic animals.

“Pharmaceutical composition” as used herein refers to a composition that includes one or more therapeutic agents disclosed herein, such as a pharmaceutically acceptable carrier, a solvent, an adjuvant, and/or a diluent, or any combination thereof.

“Therapeutically effective amount” or “effective amount” refers to that amount of a therapeutic agent, which when administered to a subject, is sufficient to effect treatment (e.g., improve symptoms) for a disease or disorder described herein, such as, for example, chronic kidney disease. The amount of a compound which constitutes a “therapeutically effective amount”, or “effective amount” can vary depending on the compound, the disorder and its severity, and the age, weight, sex, and genetic background of the subject to be treated, but can be determined by one of ordinary skill in the art.

“Treating” or “treatment” as used herein refers to the treatment of a disease or disorder described herein, in a subject, preferably a human, and includes inhibiting, relieving, ameliorating, or slowing progression of the disease or disorder or one or more symptoms of the disease or disorder.

“Subject” refers to a warm-blooded animal such as a mammal, preferably a human, which is afflicted with, or has the potential to be afflicted with one or more diseases and disorders described herein.

In view of the present disclosure, the methods and compositions described herein can be configured by the person of ordinary skill in the art to meet the desired need. In general, the disclosed contemplated composition is a formulated drug designed to supply and optimize NAD+ mitochondrial availability in order to target the loss of nephrons via renal fibrosis and tubular senescence of the kidney cells by enhancing the mitochondrial activity of these cells via the NAD/NAD+ redox pathway as well as reduce the presence of fibrogenic intracellular inflammatory markers. This therapeutic composition and methods of using the composition stabilize the intracellular oxidative phosphorylation process, which is dysregulated or abnormal causing ongoing renal tubular cell injury and damage in chronic kidney disease.

In some embodiments, the present disclosure provides pharmaceutical compositions and methods to treat chronic kidney disease (CKD). In some embodiments, contemplated pharmaceutical compositions are formulated for oral administration, injection, infusion, inhalation, or transdermal delivery.

In further embodiments, the contemplated pharmaceutical compositions include a pharmaceutically suitable carrier, dilutant, and/or excipient. The pharmaceutical compositions can also include a taste-masking agent to improve palatability for the subject. In some embodiments, the taste-masking agent is a sweetener or flavorant.

In some embodiments, the contemplated pharmaceutical compositions include a therapeutically effective amount of a coagulation modifier, a therapeutically effective amount of a ubiquinone, and a therapeutically effective amount of a nicotinamide oxygenase modifier or any NAD precursor.

In other embodiments, contemplated pharmaceutical compositions can include a therapeutically effective amount of citric acid/sodium citrate (buffer of sodium citrate and citric acid) in lieu of (or in addition to) ubiquinone. In some embodiments, a therapeutically effective amount of citric acid/sodium citrate is about 3000 mg-2004 mg, which can be adapted for level of kidney disease advancement. In some instances, the therapeutically effective amount of citric acid/sodium citrate is administered once, twice, or three times daily.

In other embodiments, contemplated pharmaceutical compositions include a therapeutically effective amount of citric acid/sodium citrate in addition to a therapeutically effective amount of ubiquinone.

In particular embodiments, the anti-inflammatory agent is pentoxifylline, the antioxidant modifier that enable intracellular supply of glutathione-a potent antioxidant is ubiquinol/ubiquinone supplied by coenzyme Q10, the NAD/NADH oxygenase modifier is acetylcysteine, which can be replaced in the formulation by direct NAD precursor, such as nicotinamide mononucleotide.

In some embodiments, a therapeutically effective amount of pentoxifylline is about 400 mg administered once daily to about 400 mg administered three times daily, which can be adapted (increased or decreased) for level of kidney disease advancement. In some embodiments, a therapeutically effective amount of CoQ10 is about 100 mg administered once daily to about 100 mg administered three times daily. In some embodiments, the therapeutically effective amount of acetylcysteine is about 1200 mg administered once daily to about 1200 mg administered twice daily. In some embodiments, the therapeutically effective amount of nicotinamide mononucleotide is about 1000 mg administered about twice daily.

Dosage amounts of the contemplated pharmaceutical compositions can be in the range of from about one tablet, powder mixture, or a mixture of pills added to a packet taken twice daily, but may be higher or lower, depending upon, among other factors, the activity of the active compound, the bioavailability of the compound, its metabolism kinetics and other pharmacokinetic properties, the mode of administration and various other factors, including particular condition being treated, the severity of existing or anticipated physiological dysfunction, the genetic profile, age, health, sex, diet, and/or weight of the subject. Dosage amounts and dosing intervals can be adjusted individually to maintain a desired therapeutic effect over time. For example, the compounds may be administered once, or once per week, several times per week (e.g., every other day), once per day or multiple times per day, depending upon, among other things, the mode of administration, the specific indication being treated and the judgment of the prescribing physician. In cases of local administration or selective uptake, such as local topical administration, the effective local concentration of compound(s) and/or active metabolite compound(s) may not be related to plasma concentration. Skilled artisans will be able to optimize effective dosages without undue experimentation.

In some embodiments, the methods include administering a therapeutically effective dose of the contemplated pharmaceutical composition to a subject to treat chronic kidney disease.

In some embodiments, the methods treat chronic kidney disease by increasing glomerular filtration rate (eGFR) and/or reducing proteinuria levels.

In some embodiments, eGFR increased in a subject by at least about 1 mL, or 2 mL, or 3 mL, or 4 mL, or 5 mL or more per minute after administration with the contemplated composition.

In some embodiments, patients have proteinuria levels in either the nephrotic or non-nephrotic range prior to treatment with the contemplated pharmaceutical composition.

In some embodiments, patients with proteinuria levels in the nephrotic range improve by at least about 5%, or 10%, or 15% or more after treatment with the contemplated pharmaceutical composition.

In some embodiments, a subject is treated with the contemplated pharmaceutical composition for at least about 1 month, or 2 months, or 3 months.

In some embodiments, a subject is treated with the contemplated pharmaceutical composition for at least about 6 months.

In some embodiments, renal function improves by at least about 5%, or 10%, or 15%, or 20% in subjects with stage 3 or stage 4 CKD when treated with the contemplated pharmaceutical composition for at least about 1 month, or 2 months, or 3 months.

In some embodiments, renal function improves by at least about 5%, or 10%, or 15%, or 20% in subjects with stage 5 CKD when treated with the contemplated pharmaceutical composition for at least about 6 months.

In some embodiments, renal function improves by at least about 5%, or 10%, or 15%, or 20% in subjects with stage 3, stage 4, or stage 5 CKD when treated with the contemplated pharmaceutical composition.

In some embodiments, the contemplated pharmaceutical composition further comprises a taste-masking agent or other flavorant as known in the art. Taste-masking agents include, but are not limited to, sweeteners, flavorants, scent blockers, and/or scent enhancers.

Various exemplary embodiments of compositions and methods according to this invention are now described in the following non-limiting Examples. The Examples are offered for illustrative purposes only and are not intended to limit the scope of the present invention in any way. Indeed, various modifications of the invention in addition to those shown and described herein will become apparent to those skilled in the art from the foregoing description and the following examples and fall within the scope of the appended claims.

### EXAMPLES

The Examples that follow are illustrative of specific embodiments of the disclosure, and various uses thereof. They are set forth for explanatory purposes only and should not be construed as limiting the scope of the disclosure in any way.

### Example 1: Treatment of CKD

### Patient Selection

All patients presenting at the clinic for their CKD management were eligible for selection. Patients with a lower eGFR at the time of screening compared to their previously measured values indicating a declining renal function were screened for participation in this study.

### Inclusion Criteria

The criteria for patients included in the study were as follows: (1) eGFR between 9 mL/min/1.72 m² and 50 mL/min/1.73 m²; (2) confirmed diagnosis of CKD; (3) estimated glomerular function by MDRD of less than 60 mL/min; (4) declining renal function (as measured by eGFR by MDRD); (5) rate of decline of eGFR over the last year of less than 20%; and (6) negative serology markers for CKD etiology.

### Exclusion Criteria

Patients were excluded from the study based on the following: (1) rapid rate of decline in kidney function of >20% over the last year; (2) symptomatic renal failure; (3) presence of any suspected acute renal failure superimposed; (4) presence of cast, hematuria, or abnormal urinalysis outside of simple UTI; and (5) no known reversible cause of renal decline.

Patients presenting in an outpatient chronic kidney disease clinic were used for the trial. Patients with slowly progressive renal function with current renal function as assessed by estimated glomerular filtration rate (eGFR) using creatinine by Modification of Diet in Renal Disease (MDRD) system were identified. Proteinuria as attained by random urine protein creatinine ratio was also obtained and documented. Patients were continuing on their standard dose of Angiotensin-converting-enzyme inhibitors (ACEIs) or Angiotensin II Receptor Blockers (ARBs) as well as use of sodium glucose cotransporter inhibitors without any subsequent change in dosage during the duration of the study.

All patients were examined and ruled out for other secondary causes of CKD. Patients with secondary causes of CKD and were removed from the trial. Causes of rapid decline in renal function with suspected superimposed acute kidney injuries were also ineligible to participate in the trial.

Regular follow up and monitoring of labs as well as side effects were documented. Standard dose of QRX3 was administered. Due to already documented major adverse effects of smell and taste, patients were encouraged to mix QRX3 with any beverage of their choice.

### Trial Design

The trial was performed as a single arm nonrandomized study. Patients were recruited from two CKD clinics in the Houston Texas area. The therapeutic composition included 400 mg of pentoxifylline, 1200 mg of acetylcysteine, and 100 mg of CoQ10, each taken up to twice daily. Patients seeking care for CKD were interviewed and screened for this trial. The trial was approved by the local clinic IRB and patient consent for drug use was obtained.

The trial was designed to enroll 20-25 patients as a phase II trial. 45 patients were screened for the trial. Initially, 20 patients were provided the therapeutic composition. The final trial resulted in a total of 17 patients, after 3 patients, that were initially included, were removed. Patients in the study had their demographics and CKD stages collected and tabulated. To eliminate the risk of acute kidney injury, eGFRs were collected from the patients 3 to 6 months before the screening and compared to renal function at the time of the trial. Patients with more than 20% rate of decline were excluded as well as those with suspected secondary acute or subacute cause of kidney injury. Proteinuria levels by random urine protein creatinine methods were documented and patients meeting nephrotic range levels as defined by random urine protein/creatinine ratio of 3 g/g/day or higher were identified and compared. Follow up data at 3 months and 6 months after start of trial intervention were collected.

### Trial Outcomes

The primary end point was the change in eGFR by MDRD for the study participants at 3 months and at 6 months compared to their baseline starting eGFR rate. eGFR was calculated from serum creatinine obtained during each visit. Random urine protein/creatinine ratio was used to determine the presence of significant proteinuria. Safety and tolerability of the drug were obtained as any reported new symptoms or events during each follow up visit and documented.

### Statistical Analysis

Analysis of the data was done with a paired two sample T-test. Baseline characteristics were evaluated by mean and standard deviations (SD) as appropriate. The percentages of increase or decrease in eGFR with the intervention at follow-up visits were calculated from the baseline eGFR values. Mean eGFR at baseline and the percentage of change at 3 months and 6 months were compared. Patients with missing appointments at 3 months but had 4 months follow up data were recorded in the 3 months data bracket. Similarly, patients with data at 7-8 months were also included in the 6 months data bracket. Patients with missing data were analyzed only with results of data they had, as we did not use any algorithm to input values for those with missing data statistical analyses were performed with the Stata 18, 2023 version (StataCorp, LLC, College Station, Texas).

A p value of 0.05 was used as the cut-off for significance.

### Results

### Patient Characteristics

Of the total 17 patients enrolled in the trial with CKD; 8 patients (47%) were in CKD stage 3. Stage 3 was divided as 3A for eGFR above 45-59 mL/min/1.72 m² and stage 3B eGFR between 30-44 mL/min/1.72 m². Two patients were in CKD stage 3A (11.7% of study population), six patients with CKD stage 3B (35.3%), 7 patients were in CKD stage 4 (41.1%) and two patients were in asymptomatic CKD stage 5 status at baseline (11.7%) (as shown in Table 1 and Table 3). Three patients with CKD stage 5 with eGFR below 15 mL/min who were initially thought to be stable but noted to have developed symptoms within two weeks of the start of the therapy were discontinued from the study and initiated on dialysis. Their data were not included in the evaluation analysis.

The majority of the patients were on ACEI/ARB (n=14, 83%) except for two patients in CKD stage 5 and one patient in CKD stage 3 who had recurrent hyperkalemia resulting in discontinuation of ACEI/ARB therapy. Eight patients were on sodium glucose lumen transporter (SGLT2) inhibitors.

TABLE 1
Baseline demographics of the study population showing
patient age, gender, common diagnoses, and CKD stage.
Demographics of study population (n = 17)

| | | |
|---|---|---|
| Average Age | 71.1 | years |
| Mean eGFR | 29.6 | mL/min |

| Male | 11 |
|---|---|
| Female | 7 |
| Diabetes mellitus (DM) | 12 |
| Hypertension (HTN) | 17 |
| Renal Artery Stenosis (RAS) | 1 |
| ACEI/ARB (Angiotensin Converting Enzyme | 14 |
| Inhibitor/Angiotensin Receptor Blocker) | |
| Sodium Glucose Lumen Transporter | 8 |
| (SGLT2) Inhibitors | |
| Stage-3 CKD | 8 |
| Stage-4 CKD | 7 |
| Stage-5 CKD | 2 |

A combination of diabetes and hypertension were the common causes of CKD in 12 of the 17 patients. Four patients had pure hypertension as their cause of kidney injury. One patient had a combination of hypertension and microvascular renal artery stenosis causing their CKD status (Table 2).

There was no age limit for participation in the trial. The mean age for this study population was 71 years with the mean eGFR of 29.4 mL per minute power 1.72-meters squared (as shown in Table 2).

TABLE 2
Shows individual patient's data = Age, clinical diagnosis causing
CKD, and corresponding estimated renal function at baseline.

| | | | Baseline eGFR (in |
|---|---|---|---|
| Patient | Age (in years) | Diagnosis | mL/min/1.72 mL/sq²) |
| | | | GFR- 0 |
| 1 | 81 | DM/HTN | 39 |
| 2 | 71 | HTN | 44 |
| 3 | 75 | HTN/DM | 30 |
| 4 | 69 | DM/HTN | 21 |
| 5 | 43 | DM/HTN | 57 |
| 6 | 74 | DM/HTN | 21 |
| 7 | 68 | HTN | 21 |
| 8 | 60 | DM/HTN | 39 |
| 9 | 83 | DM/HTN | 27 |
| 10 | 70 | DM/HTN | 17 |
| 11 | 63 | DM/HTN | 40 |
| 12 | 74 | DM/HTN | 31 |
| 13 | 79 | DM/HTN | 29 |
| 14 | 65 | HTN | 39 |
| 15 | 77 | HTN | 26 |
| 16 | 74 | DM/HTN | 9 |
| 17 | 83 | HTN/RAS | 14 |
| MEAN | 71.11765 | | 29.64706 |

GFR-0 is baseline estimated GFR at time the time of initiation. DM = Diabetes, HTN—Hypertension, DM/HTN—combined diabetes and Hypertension, RAS—Microvascular renal artery stenosis.

TABLE 3
Baseline CKD stages of Participants

| CKD | Number of | Percentage of sample |
|---|---|---|
| stage | Patients | Population |
| 3A | 2 | 11.7 |
| 3B | 6 | 35.3 |
| 4 | 7 | 41.1 |
| 5 | 2 | 11.7 |
| Total | 17 | 100% |

### Rate of Change in Kidney Function

At the time of participation in the trial, patients had a mean decline in their prior renal function; renal function from 33.8 mL/min/1.72 mL/min from the preceding 3 to 6 months prior to 32 mL/min at the time of study initiation.

Of the 17 patients, 7 (41%) patients did not have all eGFR values for the 4 time periods (Pre-treatment and 3 months and 6 months after treatment) needed for complete analysis. Some patients lacked either the post 3 or post 6 months eGFR values. Four (23.5%) of them lack the pre-baseline data but were clinically determined to be in a stable renal decline. Ten (58.8%) patients had their completed data and analysis in comparison of the pre-, baseline or 0, 3 months post and 6 months post data were done (Table 4). All of these patients had their data evaluated.

TABLE 4
Patients with completed data for all 4 time periods

| GFRPRE3 | GFR0 | GFR3 | GFR6 | |
|---|---|---|---|---|
| 43 | 57 | 81 | 81 | |
| 44 | 39 | 47 | 44 | |
| 34 | 27 | 29 | 31 | |
| 21 | 17 | 22 | 29 | |
| 42 | 40 | 54 | 48 | |
| 35 | 31 | 35 | 35 | |
| 31 | 29 | 32 | 31 | |
| 44 | 39 | 39 | 35 | |
| 11 | 9 | 10 | 10 | |
| 33.8 | 32 | 38.7 | 38.2 | Mean |
| 11.4 | 14 | 20 | 19.2 | SD |

Glomerular filtration rate changes with therapy at 3-6 months pre-initiation of study (GFR-Pre), at time of study (GFR0) and at three and six months of intervention (GFR3 and GFR6), respectively.

TABLE 5
Comparison of renal function by eGFR at baseline (GFR0),
Prior eGFR (GFRPRE3), 3 months after start of therapy
(GFR3), and 6 months after therapy initiation (GFR6).

| GFRPRE3 | GFR0 | GFR3 | GFR6 |
|---|---|---|---|
| 45 | 39 | X | 41 |
| x | 44 | 52 | 64 |
| x | 30 | 40 | x |
| X | 21 | 24 | x |
| 43 | 57 | 81 | 81 |
| 24 | 21 | 23 | x |
| 32 | 21 | 34 | x |
| 44 | 39 | 47 | 44 |
| 34 | 27 | 29 | 31 |
| 21 | 17 | 22 | 29 |
| 42 | 40 | 54 | 48 |
| 35 | 31 | 35 | 35 |
| 31 | 29 | 32 | 31 |
| 44 | 39 | 39 | 35 |
| X | 26 | 29 | 24 |
| 11 | 9 | 10 | 10 |
| X | 14 | 14 | 18 |

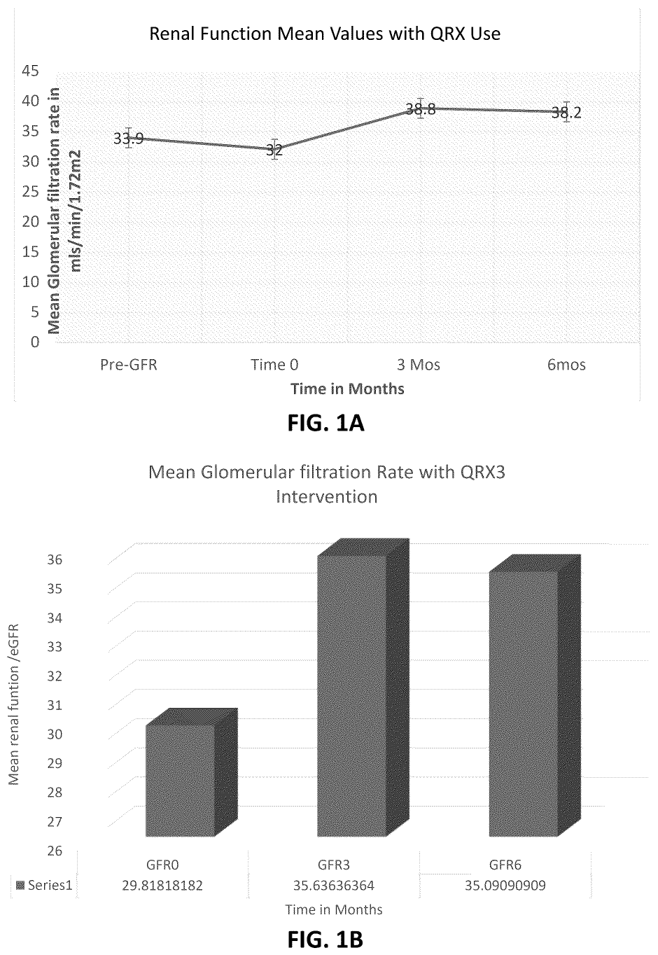

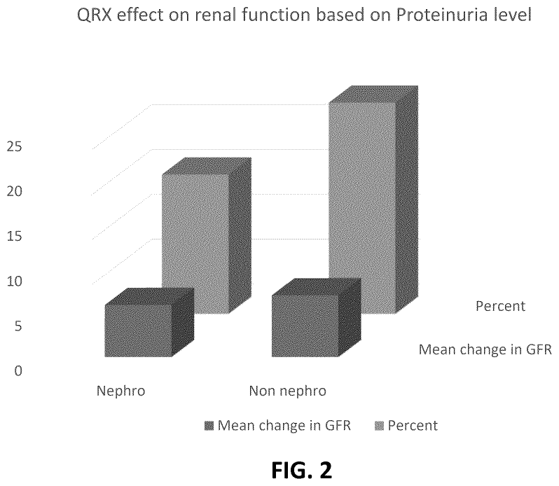

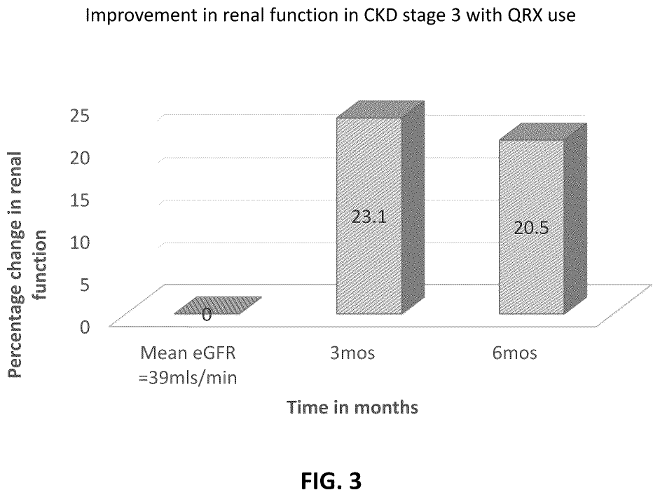

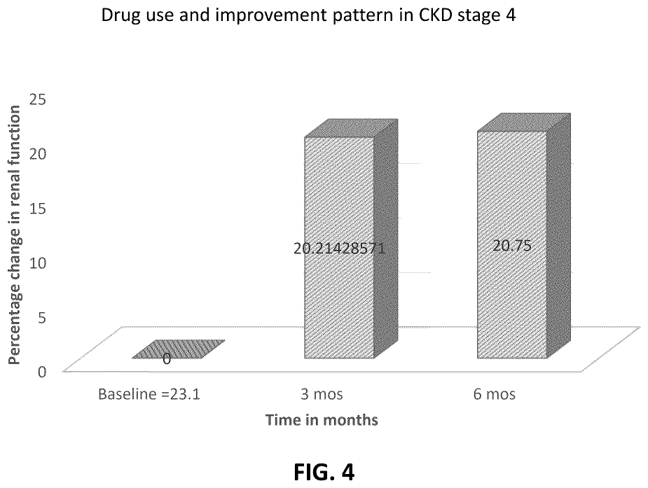

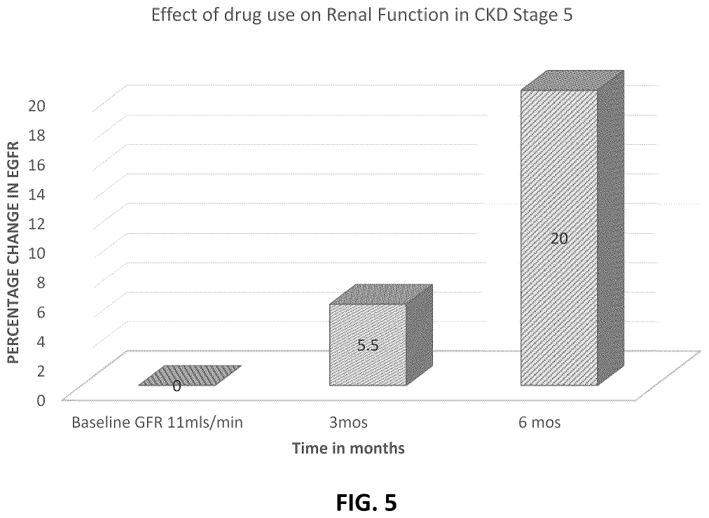

As described below and shown in FIGS. 1-5, all study participants had an increase in their renal function by use of the therapeutic composition compared to their baseline. There was a 100% increase in the renal function measured by MDRD eGFR at three months after drug initiation (Table 5). There was an average increase by 6 mL per minute from 32 mL/min at start of study to 38.8 mL/min improvement in renal function at three months. This improvement in renal function was sustained at six months with an average mean eGFR of 38.2 mL per minute P=0.04, CI 24.8-39.2).

Using analysis of the data for all participants, the mean change in renal function by eGFR was from 29 mL per minute to 35.5 mL per minute at three months (P=0.027 Confidence interval 22.68 to 35.43, DF 16, SE 3.01), which was sustained at 35.2 mL per minute at 6 months (p=0.07 DF 11 Confidence interval 23.1 to 40.05, SE 3.8). This reflected a percentage mean increase of 20.9% for all the study participants. As previously mentioned, 5 patients did not have data at 6 months follow-up.

### Effect on Proteinuria Levels

### Proteinuria Levels

Among patients with significant nephrotic proteinuria there was an average mean increase of 5.8 mL per minute (change of 15.7%, Table 6) for the nephrotic proteinuria group vs 6.7 mL per minute (change of 23.5%) for those with non-nephrotic proteinuria at the start or during the period of the trial. The nephrotic range proteinuria was defined as random urine protein creatinine ratio of greater than 3 g/g. This translated to a mean percentage increase in renal function of 15.7 percent for the proteinuria group and 23.5% for the non-proteinuria group (23.5 vs 15.7) P=0.03 CI 4.7-6.9).

TABLE 6
showing the impact of baseline proteinuria on mean change
(increased percentage above baseline) of renal function
after therapeutic composition intervention at 6 months.

| | Nephrotic Range | Non - Nephrotic |
|---|---|---|
| Mean change in GFR | 5.8 | 6.87 |
| Percent Increase | 15.5 | 23.5 |

Nephrotic range proteinuria was defined as random urine protein creatinine ratio of >3 g/g, mean change in GFR was obtained from average of all the difference between baseline and 6 months values. Percentage change from baseline values is also shown.

### Analysis of Outcome by CKD Stage

When analyzed by baseline stage patients with CKD stage 3 had an average increase of 23.1% increase in their baseline glomerular filtration function at three months which was sustained at 20.5% above the baseline value at six months (see Table 7). The mean baseline GFR for this body population was 39.8 mL per minute. For CKD stage 4, baseline eGFR was 23.1 mL/min, and there was a mean average increase of 20.2% at three months which improved to 20.7% at six months in the overall renal function.

For the 2 patients with asymptomatic CKD stage 5 there was an average increase of glomerular filtration of 0.5 mL per minute at three months which improved to 2.5 mL/minute/1.72 m2 at six months corresponding with an average increase of 5.5% at three months and at up to 20 percent at six months. None of the patients had transitioned to requiring dialysis as of this time.

The level of increase in renal function with therapeutic composition use was similar in percentage between CKD stage 3 and stage 4 but higher than the improvement in CKD stage 5 (percent increase 23.1% and 20.2% vs 5.5% (P=0.0086 for CKD 3 and P=0.02 for CKD 4). At the end of study, there was a similar overall improvement ratio (20.7 vs 20.7 vs 20%) for all CKD stages.

TABLE 7
Relationship of CKD stage with resulting level of improvement
in renal function by QRX3 drug intervention

| CKD | Mean eGFR | Percentage change | Percentage change |
|---|---|---|---|
| Stage | at baseline | at 3 months | at 6 months |
| 3 | 39.8 | 23.10% | 20.50% |
| 4 | 23.1 | 20.20% | 20.80% |
| 5 | 11.5 | 5.50% | 20% |

### Adverse Effects

12 patients (70.5%) reported poor smell and taste of the therapeutic composition that was readily ameliorated by advising mixture of the drug with a beverage. One patient (n=1, 4.7%) developed a rash that improved with removal of the P-component from the drug regimen and treatment with steroids. No other major side effect was observed. Other taste-masking or scent-masking agents can be used in place of the beverage.

### CONCLUSIONS

The disclosed composition promotes the activity of NAD+ as well minimizes the generation of inflammatory markers. Treatment with the disclosed composition resulted in an overall improvement in renal function which continued to be sustained several months after initiation of the drug.

The use of the comparison of prior renal function to the GFR status at the time of study eliminated the possibility of acute kidney injury contributing to the study outcome. The decline of average of 1.8 mL/min mean eGFR is in line with a pattern of a slow gradual progression of ongoing renal injury is typical for this study population. The results were adequate for comparing the result of the mean change in kidney function after start of intervention to the baseline GFR at time of inclusion in the study.

This is the first drug therapy that has shown any promise of reversing decline in glomerular filtration rate in patients with CKD.

The embodiments illustratively described herein suitably can be practiced in the absence of any element or elements, limitation or limitations that are not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the embodiments claimed. Thus, it should be understood that although the present description has been specifically disclosed by embodiments, optional features, modification, and variation of the concepts herein disclosed may be resorted to by those skilled in the art, and that such modifications and variations are considered to be within the scope of these embodiments as defined by the description and the appended claims. Although some aspects of the present disclosure can be identified herein as particularly advantageous, it is contemplated that the present disclosure is not limited to these particular aspects of the disclosure.

Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or all of the group members are present in, employed in, or otherwise relevant to a given product or process.

Furthermore, the disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group.

It should it be understood that, in general, where the disclosure, or aspects of the disclosure, is/are referred to as comprising particular elements and/or features, certain embodiments of the disclosure or aspects of the disclosure consist, or consist essentially of, such elements and/or features. For purposes of simplicity, those embodiments have not been specifically set forth herein.

### REFERENCES

1. Webster A C, Nagler E V, Morton R L, Masson P. Chronic kidney disease. Lancet 2017; 389:1238-1252
2. Hill N R, Fatoba S T, Oke J L, et al. Global prevalence of chronic kidney disease-a systematic review and meta-analysis. PLOS One. 2016; 11 (7): e0158765
3. Hiddo J. L. Heerspink, Ph. D et al; Dapagliflozin in Patients with Chronic Kidney Disease N Engl J Med 2020; 383:1436-144.
4. Weiner D E, Tighiouart H, Amin M G, et al. chronic kidney disease as a risk factor for cardiovascular disease and all-cause mortality: a pooled analysis of community-based studies. J Am Soc Nephrol. 2004; 15 (5): 1307-1315.
5. Tonelli M, Wiebe N, Culleton B, et al. chronic kidney disease and mortality risk: a systematic review. J Am Soc Nephrol. 2006; 17 (7): 2034-2047.
6. Chen T K, Knicely D H, Grams M E. Chronic Kidney Disease Diagnosis and Management: A Review. JAMA. 2019 Oct. 1; 322 (13): 1294-1304.
7. Mahmoodi B K, Matsushita K, Woodward M, et al. Associations of kidney disease measures with mortality and end-stage renal disease in individuals with and without hypertension: a meta-analysis. Lancet. 2012; 380 (9854): 1649-1661.
8. Go A S, Chertow G M, Fan D, McCulloch C E, Hsu C Y. Chronic kidney disease and the risks of death, cardiovascular events, and hospitalization. N Engl J Med. 2004; 351 (13): 1296-1305.
9. Ettehad D, Emdin C A, Kiran A, et al. Blood pressure lowering for prevention of cardiovascular disease and death: a systematic review and meta-analysis. Lancet. 2016; 387 (10022): 957-967.
10. Qin Y, Chen T, Chen Q, et al. The effect of angiotensin-converting enzyme inhibitor/angiotensin receptor blocker use on mortality in patients with chronic kidney disease: a meta-analysis of observational studies. Pharmacoepidemiol Drug Saf. 2016; 25 (5): 503-511
11. de Brito-Ashurst I, Varagunam M, Raftery M J, Yaqoob M M: Bicarbonate supplementation slows progression of CKD and improves nutritional status. J Am Soc Nephrol. 2009, 20 (9): 2075-2084. 10.1681/ASN.2008111205.
12. Mahajan A, Simoni J, Sheather S J, Broglio K R, Rajab M H, Wesson D E: Daily oral sodium bicarbonate preserves glomerular filtration rate by slowing its decline in early hypertensive nephropathy. Kidney Int. 2010, 78 (3): 303-309. 10.1038/ki.2010.129.
13. Phisitkul S, Khanna A, Simoni J, Broglio K, Sheather S, Rajab M H, Wesson D E: Amelioration of metabolic acidosis in patients with low GFR reduced kidney endothelin production and kidney injury, and better preserved GFR. Kidney Int. 2010, 77 (7): 617-623. 10.1038/ki.2009.519.
14. Wanner C, Inzucchi S E, Lachin J M, et al. Empagliflozin and progression of kidney disease in type 2 diabetes. N Engl J Med 2016; 375:323-334.
15. Neal B, Perkovic V, Mahaffey K W, et al. Canagliflozin and cardiovascular and renal events in type 2 diabetes. N Engl J Med 2017; 377:644-657.
16. Wiviott S D, Raz I, Bonaca M P, et al. Dapagliflozin and cardiovascular outcomes in type 2 diabetes. N Engl J Med 2019; 380:347-357.
17. Cravedi P, Remuzzi G. Pathophysiology of proteinuria and its value as an outcome measure in chronic kidney disease. Br J Clin Pharmacol. 2013 October; 76 (4): 516-23
18. Morevati M, Fang E F, Mace M L, Kanbay M, Gravesen E, Nordholm A, Egstrand S, Hornum M. Roles of NAD+ in Acute and Chronic Kidney Diseases. Int J Mol Sci. 2022 Dec. 21; 24 (1): 137
19. Genovese F., Manresa A. A., Leeming D. J., Karsdal M. A., Boor P. The extracellular matrix in the kidney: A source of novel non-invasive biomarkers of kidney fibrosis? Fibrogenesis Tissue Repair. 2014; 7:4.
20. Sparding N., Rasmussen D. G. K., Genovese F., Karsdal M. A., Hornum M., Feldt-Rasmussen B., Packington R., Selby N. M. Circulating Levels of Endotrophin Are Prognostic for Long-Term Mortality after AKI. Kidney360. 2022; 3:809-817.
21. Humphreys B. D. Mechanisms of Renal Fibrosis. Annu. Rev. Physiol. 2018; 80:309-326.
22. Jiang M., Bai M., Lei J., Xie Y., Xu S., Jia Z., Zhang A. Mitochondrial dysfunction, and the AKI-to-CKD transition. Am. J. Physiol. Renal. Physiol. 2020; 319: F1105-F1116
23. Kumakura S, Sato E, Sekimoto A, Hashizume Y, Yamakage S, Miyazaki M, Ito S, Harigae H, Takahashi N. Nicotinamide Attenuates the Progression of Renal Failure in a Mouse Model of Adenine-Induced Chronic Kidney Disease. Toxins (Basel). 2021 Jan. 11; 13 (1): 50
24. Chanvillard L, Tammaro A, Sorrentino V. NAD+ Metabolism and Interventions in Premature Renal Aging and Chronic Kidney Disease. Cells. 2022 Dec. 21; 12 (1): 21. doi: 10.3390/cells12010021. PMID: 36611814; PMCID: PMC9818486.
25. Liu X, Luo D, Huang S, Liu S, Zhang B, Wang F, Lu J, Chen J, Li S. Impaired Nicotinamide Adenine Dinucleotide Biosynthesis in the Kidney of Chronic Kidney Disease. Front Physiol. 2021 Sep. 17; 12:723690. doi: 10.3389/fphys.2021.723690. PMID: 34603081; PMCID: PMC8484911.
26. Doke T, Mukherjee S, Mukhi D, Dhillon P, Abedini A, Davis J G, Chellappa K, Chen B, Baur J A, Susztak K. NAD+ precursor supplementation prevents mtRNA/RIG-I-dependent inflammation during kidney injury. Nat Metab. 2023 March; 5 (3): 414-430. doi: 10.1038/s42255-023-00761-7. Epub 2023 Mar. 13. PMID: 36914909; PMCID: PMC10230446.
27. Isakova T., Larive B., Raphael K. L., Raj D. S., Cheung A. K., Sprague S. M., Fried L. F., Gassman J. J., Middleton J. P., et al. Effects of Nicotinamide and Lanthanum Carbonate on Serum Phosphate and Fibroblast Growth Factor-23 in CKD: The COMBINE Trial. J. Am. Soc. ephrol. 2019; 30:1096-1108.

We claim:

1. A pharmaceutical composition, comprising:
a therapeutically effective amount of an anti-inflammatory agent, a therapeutically effective amount of an ubiquinol and/or citric acid/sodium citrate, and a therapeutically effective amount of a nicotinamide oxygenase modifier or any NAD precursor.

2. The pharmaceutical composition of claim 1, wherein anti-inflammatory modifier is pentoxifylline.

3. The pharmaceutical composition of claim 1, wherein the ubiquinone/or its reduced form ubiquinol is coenzyme Q10.

4. The pharmaceutical composition of claim 1, wherein the NAD oxygenase modifier is acetylcysteine.

5. The pharmaceutical composition of claim 1, wherein the NAD precursor is nicotinamide mononucleotide.

6. The pharmaceutical composition of claim 2, wherein the therapeutically effective amount of pentoxifylline is about 400 mg to about 800 mg.

7. The pharmaceutical composition of claim 3, wherein the therapeutically effective amount of CoQ10 is about 100 mg to about 200 mg.

8. The pharmaceutical composition of claim 4, wherein the therapeutically effective amount of acetylcysteine is about 1200 mg to about 2400 mg.

9. The pharmaceutical composition of claim 5, wherein the therapeutically effective amount of nicotinamide mononucleotide is about 1000 mg to about 2000 mg.

10. The pharmaceutical composition of claim 9, wherein the composition further comprises one or more of a pharmaceutically suitable carrier, dilutant, and/or excipient.

11. The pharmaceutical composition of claim 10, wherein the composition further comprises a sweetener or a flavorant.

12. The pharmaceutical composition of claim 11, wherein the composition is a powder or mixture of pills.

13. The pharmaceutical composition of claim 12, wherein the composition is formulated for oral administration, injection, intravenous (IV), or intramuscular delivery.

14. A method of treating chronic kidney disease in a subject, the method comprising:
administering a therapeutically effective dose of a pharmaceutical composition to the subject, wherein the composition comprises,
i. a pentoxifylline;
ii. coenzyme Q10 and/or citric acid/sodium citrate; and
iii. acetylcysteine or nicotinamide mononucleotide, and
wherein the composition increases estimated glomerular filtration rate (eGFR) and/or reduces proteinuria levels in the subject.

15. The method of claim 14, wherein administration of the pharmaceutical composition improves eGFR by at least about 5 mL per minute.

16. The method of claim 14, wherein the pharmaceutical composition is delivered to the subject by oral administration, injection, intravenously, or intramuscularly.

17. A method of treating chronic kidney disease in a subject, the method comprising:
a) administering a therapeutically effective dose of a pharmaceutical composition to the subject, wherein the composition comprises,
i. about 400 mg to about 800 mg of pentoxifylline;
ii. about 100 mg to about 200 mg of CoQ10 and/or about 3000 mg-2004 mg to about 6000 mg-4008 mg citric acid/sodium citrate; and
iii. about 1200 mg to about 2400 mg of acetylcysteine or about 1000 mg to about 2000 mg of nicotinamide mononucleotide,
wherein the composition further comprises
i. one or more of a pharmaceutically suitable carrier, dilutant, and/or excipient, and
ii. a sweetener or a flavorant; and
b) at least one of increasing estimated glomerular filtration rate (eGFR) and/or reducing proteinuria levels in the subject.

18. The method of claim 17, wherein the pharmaceutical composition is administered daily for at least 3 months.

19. The method of claim 18, wherein administration of the pharmaceutical composition improves eGFR by at least 5 mL per minute.

20. The method of claim 18, wherein renal function improves by about 20% in subjects with stage 3, stage 4, or stage 5 chronic kidney disease.
